(19) United States
(12) Patent Application Publication
(10) Pub. No.: US 2020/0312463 A1
(43) Pub. Date: Oct. 1, 2020

(54) DYNAMIC HEALTH RECORD PROBLEM LIST

(71) Applicant: INTERNATIONAL BUSINESS MACHINES CORPORATION, Armonk, NY (US)

(72) Inventors: Mark D. Bronkalla, Waukesha, WI (US); David Bronkalla, Waukesha, WI (US)

(21) Appl. No.: 16/366,951

(22) Filed: Mar. 27, 2019

(51) Int. Cl. G16H 50/70; G16H 50/20; G06F 17/28

(52) U.S. Cl. CPC G16H 50/70; H04L 67/12; G06F 17/28; G16H 50/20

(57) ABSTRACT

Methods and systems generate a health record problem list. One system includes an electronic processor configured to obtain electronic health data, apply natural language processing to generate extracted health data from the electronic health data, and map the extracted health data to normalized terms in one or more ontologies, thereby generating normalized health data. The electronic processor is also configured to determine if any relationship exists between the normalized health data, and, when a relationship exists between the at least two medical record entries using an ontological list, generate a relationship map relating the originating medical data with the subsequent relevant medical data. The electronic processor is also configured to receive a request for dynamic health record problem list data from a client device, and provide the dynamic health record problem list data to the client device.

### FIELD OF DISCLOSURE

The present disclosure relates to medical health record data. More particularly, the present disclosure relates to systems and methods for analyzing and presenting information relating to medical health record data.

### SUMMARY

Many electronic medical records (EMRs) include a “problem list” for a patient. Typically, the problem list is manually entered and manually maintained. The problem list provides a quick summary of issues for a given patient. Usually, each item in the problem list is a brief (two to four word) description. However, these descriptions often lack information regarding why an item was entered, when the item was entered, whether the item has been resolved, any relationships between items, or the like. Thus, many EMRs have a problem list that is merely an uncategorized, unsorted list. During a typical patient visit, clinicians often view the problem list for the patient first but an uncategorized, unsorted list fails to provide clinicians with an accurate overview of the patient. When referring to EMR, this also covers other electronic records such as Hospital Information System (HIS), Personal Health record (PHR) or public health records as well.

For example, some institutions do not remove items from the problem list because these items may be a basis for listing additional comorbidities for a patient or may impact insurance reimbursement or other administrative reasons. However, the number of items in the problem list adds to the “noise” for a clinician's review. Thus, it can be difficult for a clinician to determine an overview of a patient's condition. This forces the clinician to click through various notes in the EMR and often give up hope of finding the source of a problem list reference. This manually clicking and navigation may be thought of as the equivalent to “flipping through the chart” when using paper charts. The issue is exacerbated by information being compartmentalized within the EMR. For example, a clinician may struggle to determine if the problem list item was originally derived from information in a clinic visit note or physical exam, imaging exam, a CCDA from another institution or another category, and, in many situations, the clinician may need to manually search each category within the EMR.

Clinicians can benefit from being able to trace issues in a patient's problem list back to the origin of the finding. This can be challenging particularly with longer problem lists and problem lists with chronic diseases (e.g. chronic systolic heart failure, various cancers, anemia, atrial flutter, GI hemorrhage, etc.). Tracing the issue to the origin can be useful in assessing whether a problem contradicts current evidence (e.g., is atrial flutter still a problem after a recent pacemaker implant?), identifying how serious or serve a condition is, or trends in a problem (e.g. heart failure with LVEF dropping from 50% to 30% in 6 months).

Thus, it can be beneficial to view each problem as being similar to a story line in a novel (the novel being the patient's history). To effectively treat a particular problem, a clinician can traverse that story line looking for notable events such as the initial detection, treatments (whether successful or not), transitions in the problem, progression of the problem or, in some cases, the resolution of the problem. For many patients there are multiple problems and a long history of the problems. Managing the interconnections of the problems is a more difficult task as the patient's problem list increases.

Accordingly, aspects of the instant disclosure are directed to generation and presentation of a dynamic health record problem list. As used herein, a “dynamic health record problem list” includes an interactive interface enabling a user to explore relationships between electronic health data for a patient. Aspects of the instant disclosure also can include linking the problem list to the source material, ranking of the source and its type, indicating one or more patient trends, making suggestions, and other aspects described in greater detail herein.

In particular, embodiments described herein provide a system for generating a health record problem list. The system includes an electronic processor and memory storing instructions that, when executed by the electronic process, cause the system to obtain electronic health data, apply natural language processing to generate extracted health data from the electronic health data, map the extracted health data to normalized terms in one or more ontologies, thereby generating normalized health data, determine if any relationship exists between the normalized health data, when a relationship exists between the at least two medical record entries using an ontological list, generate a relationship map relating the originating medical data with the subsequent relevant medical data, receive a request for dynamic health record problem list data from a client device, and provide the dynamic health record problem list data to the client device, wherein the dynamic health record problem list data includes the relationship map. Determining if any relationship exists between the normalized health data can include identifying originating medical issue data and identifying subsequent relevant medical data.

Another embodiment provides non-transitory computer-readable medium including instructions that, when executed by an electronic processor, perform a set of functions. The set of functions includes obtaining electronic health data, applying natural language processing to generate extracted health data from the electronic health data, mapping the extracted health data to normalized terms in one or more ontologies, thereby generating normalized health data, determining if any relationship exists between the normalized health data, which can include identifying originating medical issue data and identifying subsequent relevant medical data, when a relationship exists between the at least two medical record entries using an ontological list, generating a relationship map relating the originating medical data with the subsequent relevant medical data, and generating a practice recommendation based on the normalized health data.

A further embodiment provides a method for generating an interactive patient data problem list with a server. The method includes obtaining electronic health data, applying natural language processing to generate extracted health data from the electronic health data, mapping the extracted health data to normalized terms in one or more ontologies, thereby generating normalized health data, determining if any relationship exists between the normalized health data, which can include identifying originating medical issue data and identifying subsequent relevant medical data, when a relationship exists between the at least two medical record entries using an ontological list, generating a relationship map relating the originating medical data with the subsequent relevant medical data, receiving a request for dynamic health record problem list data from a client device, and providing the dynamic health record problem list data to the client device, wherein the dynamic health record problem list data includes the relationship map.

Other aspects of the disclosure will become apparent by consideration of the detailed description and accompanying drawings. There is no specific requirement that a material, technique or method include all of the details characterized herein, in order to obtain some benefit according to the present disclosure. Thus, the specific examples characterized are meant to be exemplary applications of the techniques described, and alternatives are possible.

### BRIEF DESCRIPTION OF THE DRAWINGS

FIG. 1 illustrates an example system for generating a dynamic health record problem list.

FIG. 2 is a flowchart illustrating an example method for generating a dynamic health record problem list performed by the system of FIG. 1.

FIG. 3 is a flowchart illustrating an example method for presenting a dynamic health record problem list performed by the system of FIG. 1.

FIG. 4 illustrates an example problem list for a patient.

FIG. 5 illustrates an example relationship map for a dynamic health record problem list.

FIG. 6 illustrates an example initial interface presenting a dynamic health record problem list.

FIG. 7 illustrates an example expanded interface showing a portion of a dynamic health record problem list shown in FIG. 6.

FIG. 8 illustrates an expanded view of the interface shown in FIG. 7.

FIG. 9 illustrates an expanded view of the interface shown in FIG. 8.

FIG. 10 illustrates an interface including an example notice.

FIG. 11 illustrates an interface including an alternative example notice.

FIG. 12 illustrates an alternative example of an initial interface that is present in the EMR/PHR rather than natively within this application.

FIG. 13 illustrates an expanded view interface based on user interaction with FIG. 12.

### DETAILED DESCRIPTION

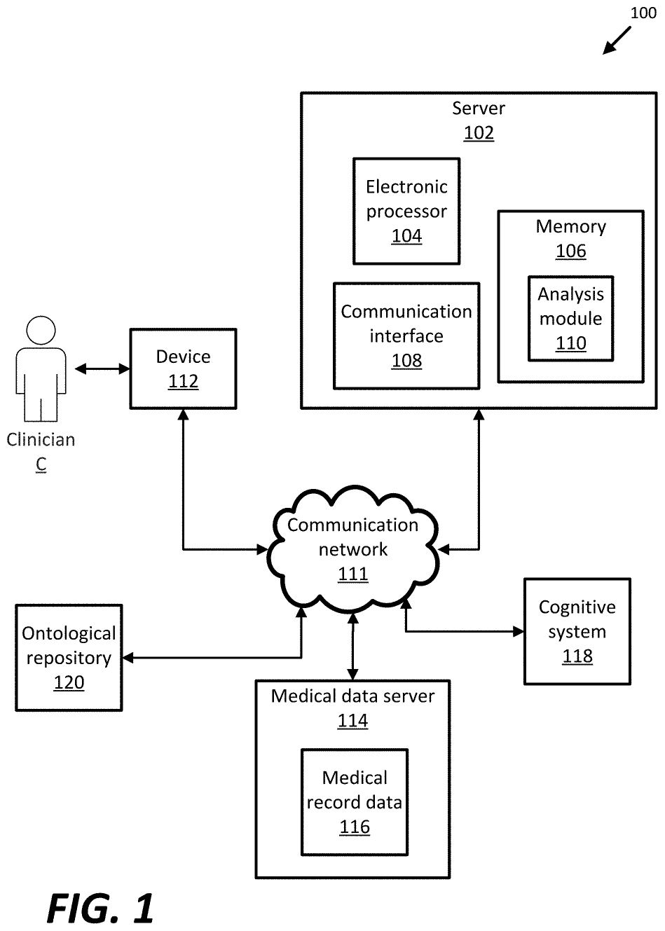

FIG. 1 illustrates an example system 100 for generating a dynamic health record problem list. The system 100 includes a server 102, a device 112, a medical data server 114, a cognitive system 118, and an ontological repository 120. Various components of the system 100 communicate via communication network 111. Clinician C interacts with device 112 to access various aspects provided by an analysis module 110, which is hosted on server 102. Other embodiments of the system 100 can include more, fewer, or different components.

The server 102 includes a plurality of electrical and electronic components that provide power, operational control, and protection of the components within the server 102. For example, as illustrated in FIG. 1, the server 102 includes an electronic processor 104 (one or more of a microprocessor, application-specific integrated circuit (ASIC), or another suitable electronic device), a memory 106 (one or more non-transitory, computer-readable storage mediums), and a communications interface 108. The electronic processor 104, the memory 106, and the communications interface 108 communicate over one or more connections or buses.

It should be understood that the server 102 illustrated in FIG. 1 represents one example of a server and embodiments described herein may include a server with additional, fewer, or different components than the server 102 illustrated in FIG. 1. In some embodiments, the server 102 performs functionality in addition to the functionality described herein. Similarly, the functionality performed by the server 102 (through execution of instructions by the electronic processor 104) may be distributed among multiple servers (including servers included a cloud-based computing system or service). Accordingly, functionality described herein as being performed by the electronic processor 104 may be performed by one or more electronic processors included in the server 102, external to the server 102, or a combination thereof.

The memory 106 may include read-only memory (ROM), random access memory (RAM) (for example, dynamic RAM (DRAM), synchronous DRAM (SDRAM), and the like), electrically erasable programmable read-only memory (EEPROM), flash memory, a hard disk, a secure digital (SD) card, other suitable memory devices, or a combination thereof. The electronic processor 104 executes computer-readable instructions (“software”) stored in the memory 106. The software may include firmware, one or more applications, program data, filters, rules, one or more program modules, and other executable instructions. For example, the software may include instructions and associated data for performing the methods described herein. For example, as illustrated in FIG. 1, the memory 106 may store the analysis module 110 (for example, software) for performing medical record analysis as described herein. It should be understood that the functionality described herein as being performed by the analysis module 110 may be distributed among multiple software modules, hardware components, or a combination thereof stored or included in the server 102 or external to the server 102.

The communications interface 108 allows the server 102 to communicate with devices external to the server 102. For example, as illustrated in FIG. 1, the server 102 may communicate with the device 112 and the medical data server 114. In particular, the communications interface 108 may include a port for receiving a wired connection to an external device (for example, a universal serial bus (USB) cable and the like), a transceiver for establishing a wireless connection to an external device (for example, over one or more communication networks 111, such as the Internet, a local area network (LAN), a wide area network (WAN), and the like), or a combination thereof. It should be understood that FIG. 1 illustrates one example of the system 100 and, in some embodiments, the server 102 may communicate with fewer or additional systems and components than illustrated in FIG. 1. For example, the server 102 may be configured to communicate with multiple medical data servers 114, multiple devices 112, or a combination thereof.

Systems and components illustrated in FIG. 1 may be combined and distributed in various configurations. For example, in some embodiments, the server 102 may include the cognitive system 118, the ontological repository 120, or a combination thereof. In some embodiments, the server 102 may also communicate with one or more user devices 112 (terminals, tablet computers, laptop computers, desktop computers, smart wearables, smart televisions, and the like) that include similar components as the server 102. For example, in some embodiments, a user may interact with the server 102 via a user device 112 to configure the system 100, such as by configuring or customizing the functionality of the server 102 as described herein. Although not illustrated in FIG. 1 or described herein, the device 112, the medical data server 114, the cognitive system 118, and the ontological repository 120 may include similar components as server 102.

The ontological repository 120 stores ontological data, such as relationship data for medical events. For example, in some embodiments, the ontological repository 120 includes mapping data relating and weighting different possible entries in medical records. Example mapping data can be stored, for instance, as one or more look-up tables, as two-dimensional maps, and as three-dimensional maps.

The cognitive system 118 is a computer system that applies machine learning (artificial intelligence) to mimic cognitive functions, including but not limited to learning and problem solving. Machine learning generally refers to the ability of a computer program to learn without being explicitly programmed. In some embodiments, a computer program (sometimes referred to as a learning engine) is configured to construct a model (for example, one or more algorithms) based on example inputs. Supervised learning involves presenting a computer program with example inputs and their desired (actual) outputs. The computer program is configured to learn a general rule (a model) that maps the inputs to the outputs.

The computer program may be configured to perform machine learning using various types of methods and mechanisms. For example, the computer program may perform machine learning using decision tree learning, association rule learning, artificial neural networks, inductive logic programming, support vector machines, clustering, Bayesian networks, reinforcement learning, representation learning, similarity and metric learning, sparse dictionary learning, and genetic algorithms. Using some or all of these approaches, a computer program may ingest, parse, and understand data and progressively refine models for data analytics. Once trained, the computer system may be referred to as an intelligent system, an artificial intelligence (AI) system, a cognitive system, or the like. Accordingly, in some embodiments, the cognitive system 118 includes Watson® provided by IBM Corporation. The cognitive system 118 may be “trained” using various machine learning techniques. In some embodiments, the cognitive system 118 may be trained using a training set of dynamic health record problem lists.

Rather than simply replicating and speeding existing human processes, computers may simultaneously process multiple tasks and draw upon multiple simultaneous information sources based on interactive rules. Therefore, unlike the human brain, which is largely a serial processor, multi-tasking computer system may simultaneously weigh many factors, and therefore complement or exceed human performance with regard to medical record interpretation.

As illustrated in FIG. 1, the server 102 may also communicate with the medical data server 114. In some instances, the server 102 communicates with multiple medical data servers 114, where each medical data server 114 is associated with a different entity, such as different providers, health collaboratives, HMOs, etc. The medical data server 114 includes one or more storage devices that include medical record data 116. The medical record data 116 can be referred to herein as electronic health record (EHR) data, electronic medical record (EMR) data, or personal health record (PHR) data.

The server 102 may also communicate with a device 112 via the communication network 111. Broadly, clinician C uses device 112 to interact with analysis module 110 and/or data generated by analysis module 110. Device 112 is a computing device that clinician C can access in a clinical setting and/or outside of a clinical setting. Examples of device 112 include a smart phone, a tablet computer, a desktop computer, a wearable device, a laptop, and the like. The device 112 can include software configured to communicate with the analysis module 110 and perform one or more display operations disclosed and contemplated herein.

Device 112 may include other software applications. Example software applications may enable clinician C to display an EMR, a PACS system or other system which can then be used to provide a contextual link and launch of the user interface for systems described herein. The linking could be programmatic or via a UI “scraping” of the other application. That is, dynamic health record functionalities described herein can be provided via a freestanding UI or linked into, or embedded within, the UI of another larger system such as that of the EMR.

The server 102 can be configured to use natural language processing to extract concepts from unstructured medical documentation stored in the medical record data 116. Example data included in the medical record data 116 includes imaging reports, visit notes, surgical notes, discharge summaries, etc. A commercially available solution for such activities has been demonstrated as part of the IBM Watson Health Patient Synopsis and Clinical Review. The server 102 can also align the extracted concepts with categories such as HPI (History of Present Illness), social history (personal history), allergies, medications, current findings, etc.

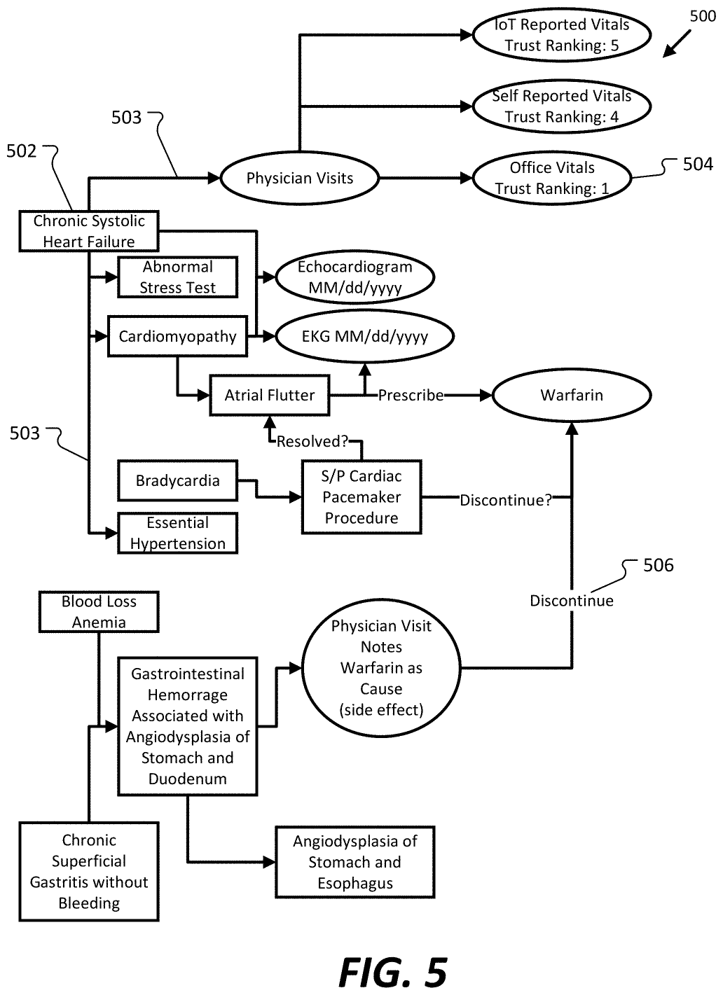

The server 102 can be configured to present the data, or cause the data to be presented on device 112, in a summary form so that the clinician can get an overview of the patient's condition. For example, a problem list can be presented as part of a user interface displayed on device 112. The displayed interface is interactive. For example, a clinician can expand and contract the view to show more or less data relating to a particular issue in the problem list. Example arrangements and configurations are described below and shown in, at least, FIGS. 5-13.

The server 102 can be configured to normalize terminology from extracted text. The server 102 can use ontologies such as the unified medical language system (UMLS) to map the extracted text and to terminology used in the ontology. In some instances, the mapped text from the ontology can be mapped to another ontology, such as the Core Problem List subset of Systematized Nomenclature of Medicine—Clinicial Terms (SNOMED-CT).

In some instances, clinicians do not perfectly adhere to the Core Problem List, so there may be terms in a problem list that are not an exact match for the Core Problem List subset. When matching the clinical terms or concepts, in some instances there is no expectation that exact matches are required. Within the ontologies, there are lists of synonyms and hierarchical relationships. Matching may be done at the same level of specificity or use the hierarchical relationships to provide better matching (e.g. hand is part of arm or simplistically convert from a brand name to generic medication). In order to provide better matching across the ontologies, it may be necessary to add additional synonyms to one ontology or the other as well as train the AI components to provide fuzzy matching that is able to conquer inconsistencies in terminology. Without the ability to match with multiple characteristics or levels of specificity across ontologies, the ability to create linkages becomes limited. Additionally other references such as for medication interactions such as Micromedex™ or Cerner Multum™ or other external reference services could be used for linkage definitions.

The server 102 can be configured to de-duplicate and/or rank the source of truth. Typically, not every entry in a patient's problem list deserves equal weight. Additionally, there may be later confirmatory tests that take precedence or show one or more trends. For example, when a patient goes to a cardiologist for a second opinion or referral for “shortness of breath” and “heart failure,” this visit could hit the problem list immediately, but further testing, such as a metabolic panel, EKG, echocardiogram and stress test, can provide further confirmatory or contradictory data. In this case, the echocardiogram and stress test reports would provide the confirmation. The server 102 can be configured to correctly categorize the problem and de-duplicate problems—removing shortness of breath from the problem list and replacing shortness of breath with heart failure. In this example, the stress test results would be the primary diagnostic test and ranked highest for confirmation of the heart failure problem. The echocardiogram test data would be ranked next and so on, back to the verbal complaint. This example illustrates that the oldest reference to the problem is not necessarily the best reference, because the original entry lacked diagnostic specificity (or was a self-reported issue or condition) and can, in some cases, be quite inaccurate in describing or diagnosing the condition. However, it is likely useful to retain the linkage, so that when the patient refers to or makes a similar complaint in the future it can be shown how this had been diagnosed or resolved in the past.

The server 102 can also be configured to deprioritize resolved and/or dormant issues. As noted above, problem lists can include findings or health issues that are no longer present or have been treated. De-prioritization can present the problem list more efficiently to providers by showing the relevant problems first but still showing the resolved/dormant issues as a low priority (retaining the resolved/dormant issues for historic reference but placing less emphasis on these issues).

As an example of deprioritizing, a patient's EMR portal may show that a history of supraventricular tachycardia (SVT) is present. SVT may be a relevant health issue for providers but, in this example, an ablation was previously performed and there has been no reoccurrence in 8 years. Server 102 uses natural language processing to extract the report of the ablation. Then server 102 can de-prioritize the health issue using the extracted ablation report. The report from the ablation procedure can also be supplemented with patient confirmation that the issue has not re-presented itself. Another perhaps more obvious example is a patient who presents for a broken arm, the fracture is on the current problem list for some period of time but then has been shown to be healed in a follow up visit note. Another variant might be that the follow up and resolution of the problem was done with another physician in an unrelated health system and the problem can either be auto resolved or prompt for resolution on the next visit. This would be a common case when dealing with problems of “snowbirds” who get part of their care in their northern/home state and receive part of their care in the winter in their southern/vacation home state.

The server 102 can also be configured to link items in a problem list to one or more source documents. Linking items can provide the clinician with the ability to filter and aggregate events using various attributes. Example attributes include visit, provider, indications, and document type.

As an example, an issue in the problem list may surface via a visit note conclusion or an imaging report. These entries would have a relatively direct tie to the problem list. However, there are related events that may or may not be mentioned in the visit notes, such as medication prescriptions, orders for physical therapy, and other imaging studies. Documents and notes can be grouped by various relationship sets, such as temporal (within a certain number of hours or days), admission or encounter-based (orders placed in the same visit or encounter), based on the ordering or prescribing clinician, or the like. Thus, events can be filtered and aggregated despite a patient seeing multiple practitioners or specialties in a single visit (e.g., a patient that sees both an orthopedic physician and an ophthalmologist on the same day).

The server 102 can also present the dynamic health record problem list as a textual or graphical timeline. In some instances, timelines can be generated based on a particular problem selected in the user interface. The server 102 can also present timelines based on a degree of specificity of the linkage based on one or more variables, such as direct linkage, indirect linkage, date correlation, physician, etc.

The server 102 can also retrieve and/or process data from medical data server 114 in response to a triggering event or pre-processed according to a schedule. That is, in some embodiments, processing of electronic medical record data and generation of dynamic health record problem lists occurs based on a triggering event. Example triggering events include patient admission, a scheduling, and an ordering event. In some embodiments, processing and dynamic health record problem list generation occurs based on a schedule.

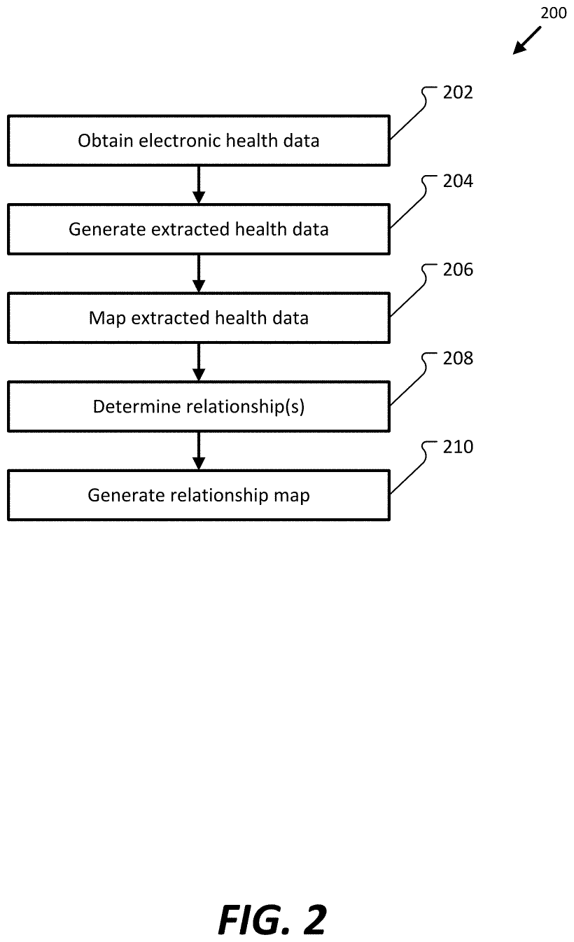

FIG. 2 shows example method 200 for generating a dynamic health record problem list. In some embodiments, as discussed above, the server 102 can be configured to execute one or more operations of method 200. Method 200 includes obtaining electronic health data (operation 202), generating extracted health data (operation 204), mapping extracted health data (operation 206), determining one or more relationships (operation 208), and generating a relationship map (operation 210). Other embodiments can include more or fewer operations.

Method 200 begins by obtaining electronic health data (operation 202). Obtaining electronic health data (operation 202) can include communicating with one or more remote servers to request and receive electronic medical record (EMR) data and/or electronic health record (EHR) data. In some instances, the remote servers are operated by separate institutions. Example electronic health data include imaging reports, visit notes, surgical notes, discharge summaries, test data, etc. Typically, much of the electronic health data are unstructured with limited amounts of structured or coded data intermingled.

After obtaining the electronic health data (operation 202), extracted health data is generated (operation 204). Generating extracted health data (operation 204) can include applying natural language processing to yield machine-readable text. In some instances, cognitive system 118 includes a natural language processing engine configured to perform natural language processing actions. Generating extracted health data (operation 204) can include optical character recognition (OCR) operations depending upon the nature of the files in the electronic health data. Natural language processing can be performed to extract concepts using methods known in the art.

After generating extracted health data (operation 204), the extracted health data is mapped (operation 206) to normalized terms in one or more ontologies. Mapping extracted health data (operation 206) includes leveraging cognitive system 118 to perform the mapping analysis. In some instances, there may be one or more periods where cognitive system 118 is trained using training data. Various ontologies can be used during operation 206, such as UMLS and SNOMED-CT or proprietary databases such as Micromedex.

After mapping extracted health data (operation 206), one or more relationships among the data are determined (operation 208). Typically, determining relationships (operation 208) is performed by cognitive system 118. As an example, determining relationships (operation 208) can include identifying, for each issue or entry in the extracted health data, which other issues or entries are related to that entry, a relative timeline, whether there are subsequent actions or entries addressing or resolving that entry. The primary interactions extracted from the ontologies include but are not limited to: manages, treats, complicates, interacts with, produces, causes and the “physically related to” group. In some instances, determining relationships (operation 208) includes identifying connections that require clinician follow-up, such as identifying unresolved, unidentified, or unaddressed medical issues.

In some instances, determining relationships (operation 208) can include determining various types of rankings for one or more entries in the extracted health data, such as accuracy rankings and veracity rankings. Various rankings can be based on whether medical data were gathered via a regulated medical device or medical personnel with medical device certification. Rankings can also be based on the source of data, such as clinician measured, auto monitored, home monitored, self-reported, and the like.

Based on relationships generated during operation 208, the server 102 can generate a relationship map (operation 210). In some instances, the relationship map can be stored in a database accessible by server 102 and/or device 112. Generally, the relationship map includes data about connections between entries in the problem list and data usable for displaying the map on a user interface. The problem list displayable to a user includes the extracted health data, the relationships, and the relationship map.

In some instances, method 200 can additionally include checking for updated medical record data. Checking for updated medical record data can include communicating with one or more medical data servers and requesting updated data, comparing the updated data to data already analyzed by analysis module 110, and generating an updated relationship map. Checking for updated medical record data can be on demand or based on predetermined intervals, such as hourly, daily, weekly, or monthly or triggered by external or messaging events such as an Admission (HL7 ADT), Scheduling/appointment (HL7 SIU), Order (HL7 ORM), Report (HL7 ORU or MDM) or other messages.

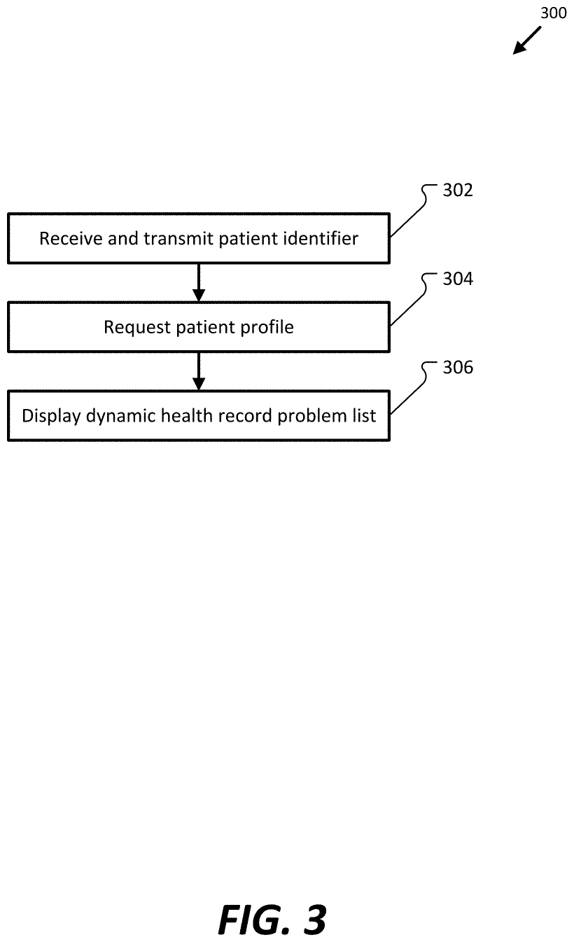

FIG. 3 shows example method 300 for presenting a dynamic health record problem list. A user device, such as device 112 shown in FIG. 1, typically performs operations shown in method 300. During an exemplary patient visit, a clinician signs in or logs onto a computing device. That process can include providing identifying information, such as a user name, as well as one or more passwords. Typically, the clinician's profile is associated with one or more patient appointments for that day and the clinician can select the profile associated with a particular patient in the room. That is, the device 112 receives a patient identifier and then sends that patient identifier (operation 302) to a remote server, such as the server 102 shown in FIG. 1.

After verifying the patient with the server (operation 302), the device 112 can request and receive the patient profile (operation 304). The patient profile can include a problem list and/or a dynamic health record problem list generated using the systems and techniques disclosed in this document. Upon receiving the dynamic health record problem list, the device 112 can display the dynamic health record problem list (operation 306) on a graphical user interface (GUI).

Displaying the dynamic health record problem list (operation 306) can include receiving user interaction with the GUI. For instance, the device may receive a selection of a particular health issue and a reverse walk for that particular issue is then displayed. In some instances, the patient profile received during operation 304 includes data sufficient for the device 112 to filter and selectively display paths and parts of the patient's problem list. In other instances, after receiving a user selection, the device may request additional data from the server, and then display received data on the GUI.

### EXAMPLE IMPLEMENTATIONS

In the following sections, various example implementations are provided, without limitation, to illustrate various aspects of the disclosed systems and methods.

### Example 1

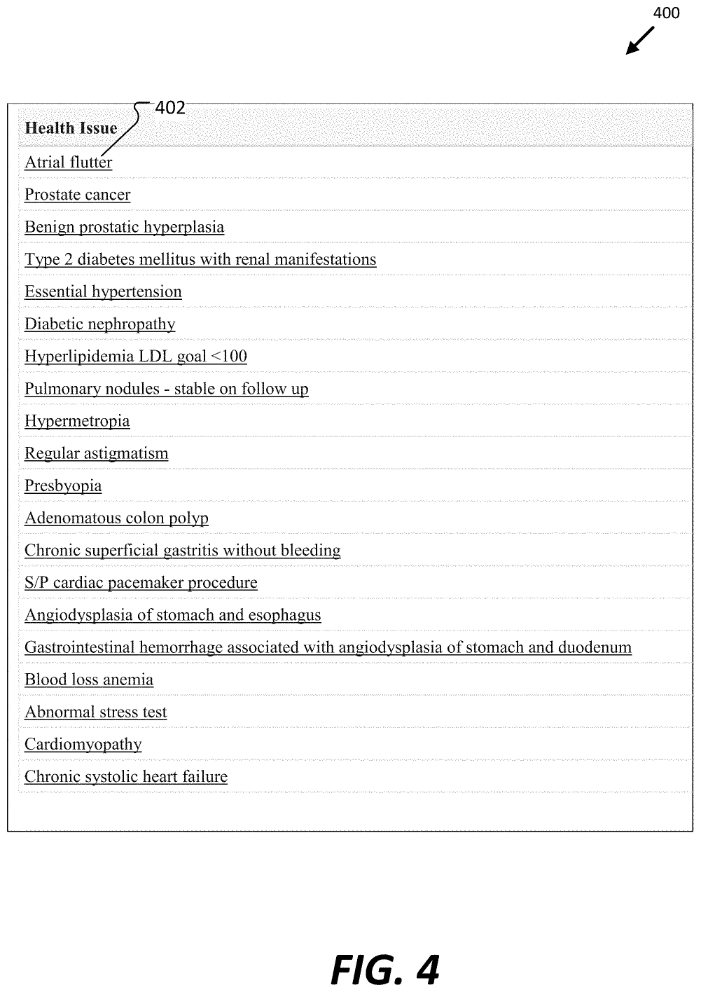

FIG. 4 shows an example problem list 400 including a plurality of health issues 402 that is shown in a Personal Health Record or EMR. Each health issue 402 is a short description in the case of the PHR and is linked to a portal that provides generic information about the problem and its treatment for the patient with generic explanations of the terms. In the portal, about 70% of the items had dates and there were a few additional items removed as part of the de-identification. However, the portal did not include any indication whether the health issues were current or had been resolved. Additionally, the problem list 400 does not include current findings or test results.

FIG. 5 shows a portion of example dynamic health record problem list 500 for problem list 400 shown in FIG. 4. The dynamic health record problem list 500 can be generated using systems and methods disclosed herein. Other portions of dynamic health record problem list 500 could be viewable upon selection by a clinician of a health issue 402. As shown, dynamic health record problem list 500 includes medical record entries 502 connected by paths 503.

In the example shown, the patient has bleeding in the stomach and esophagus that has progressed from blood loss anemia and angiodysplasia of the stomach. The initial cause was determined to be warfarin. But the system also then linked the atrial flutter diagnosis, where warfarin was prescribed as a treatment to prevent stroke (due to potential for clot formation form the atrial flutter). Warfarin was the link between the two problems or events.

Some paths 503 include a practice recommendation 506. In some instances, cognitive system 118 generates practice recommendation 506 using one or more ontologies and best practice guidelines. As examples, practice recommendation 506 can be a suggestion, such as a clinical determination (e.g., “discontinue”) or a flag for follow up (“resolved?”).

Certain medical record entries 504 can include trust rankings. Generally, a trust ranking provides an indication of the veracity of the data. Trust rankings can be based on various factors, such as the information source. For instance, self-reported vital signs such as blood pressure may be given a lower trust ranking (4) than vital signs obtained in a clinical setting (1).

In some embodiments, the dynamic health record problem list 500 displays medical record entries 502 in order of priority (e.g., primary, secondary, tertiary). For instance, medical record entries 502 having primary treatment priority can be displayed at the top of the screen, followed by entries with secondary importance, and then followed by entries with tertiary importance.

In some embodiments, the medical record entries 502 are selectable. For instance, upon selection of a medical record entry 502, the user interface displays additional data relating to medical record entry 502.

### Example 2

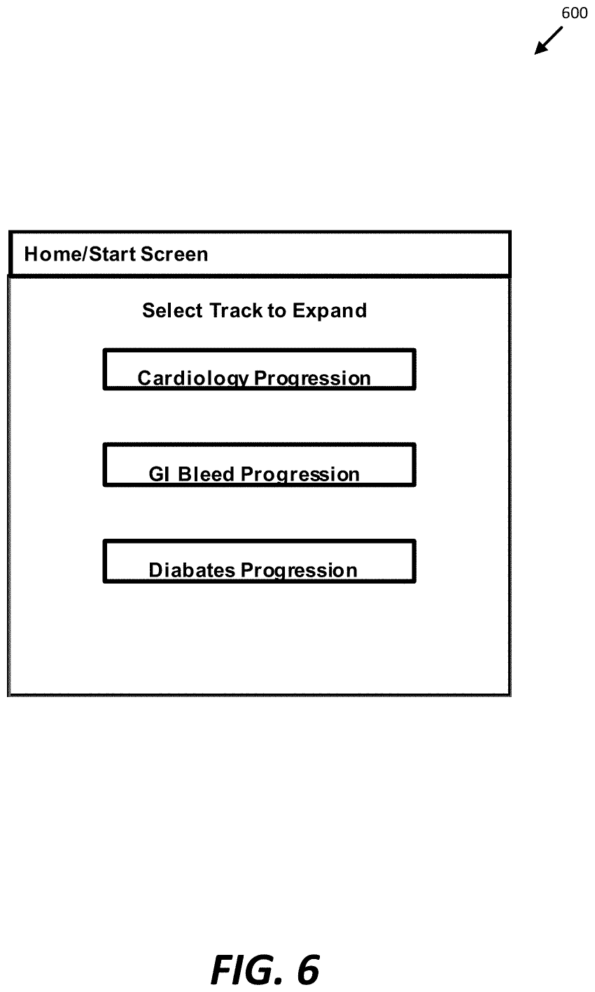

FIGS. 6-13 show various example graphical user interfaces that may be displayed on a user device, such as device 112. FIG. 6 shows interface 600 as an example initial screen to be shown upon application launch. In some instances, interface 600 is part of a program that is launched by device 112 and is not hosted on a remote server or electronic medical record server. Additionally, a remote server, such as server 102, has already pre-processed the patient's electronic medical record data to generate the dynamic health record problem list.

As part of that pre-processing, the server may generally group entries in the dynamic health record problem list by type. As shown in FIG. 6, the server has identified three tracks: a cardiology progression, a GI bleed progression, and a diabetes progression.

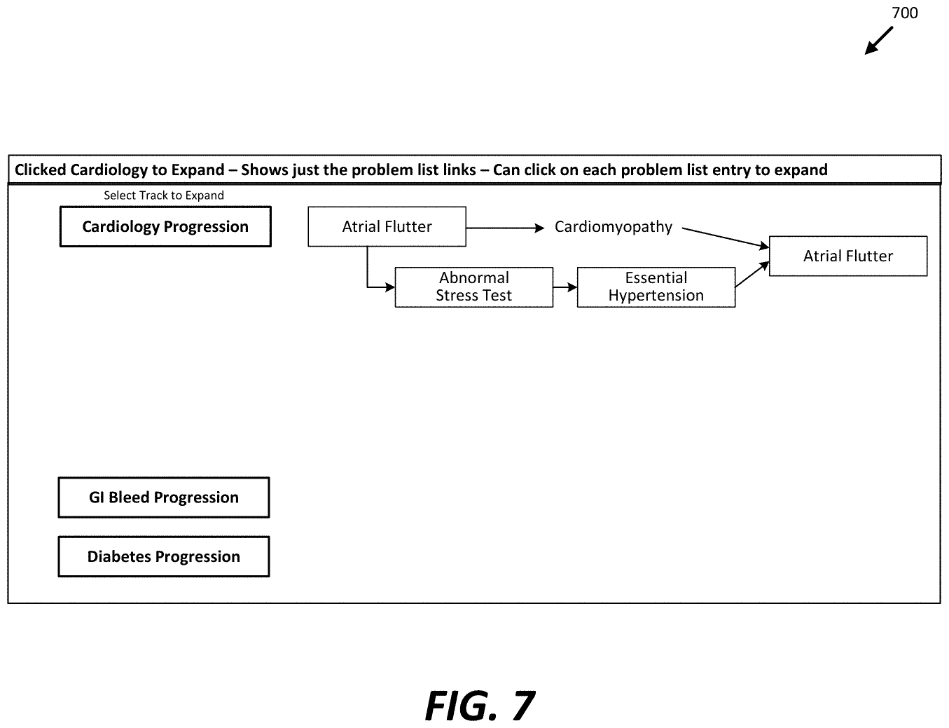

FIG. 7 shows interface 700, which may be displayed after a user selects the “Cardiology Progression” track. Interface 700 shows problem list entries that were linked together under the ontology. Interface 700 also shows a progression through time, where a problem list entry may have expanded into another entry.

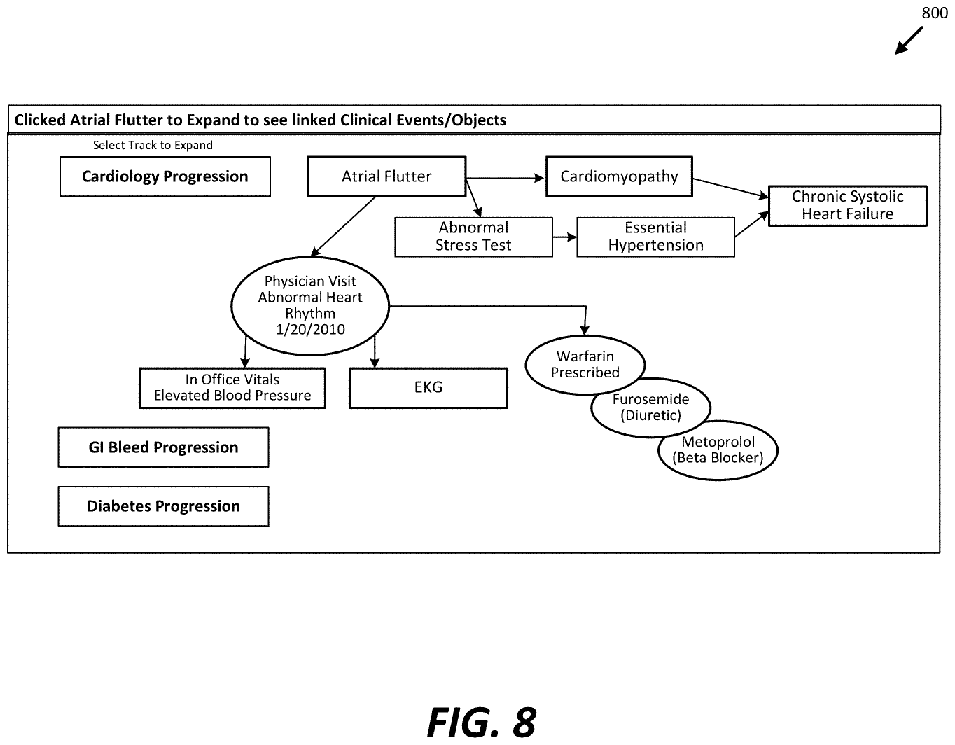

FIG. 8 shows interface 800, where the user has selected to expand one issue in the problem list in interface 700. Interface 800 shows the links from the selected issue to physician visits, procedures, reports, prescriptions, etc.

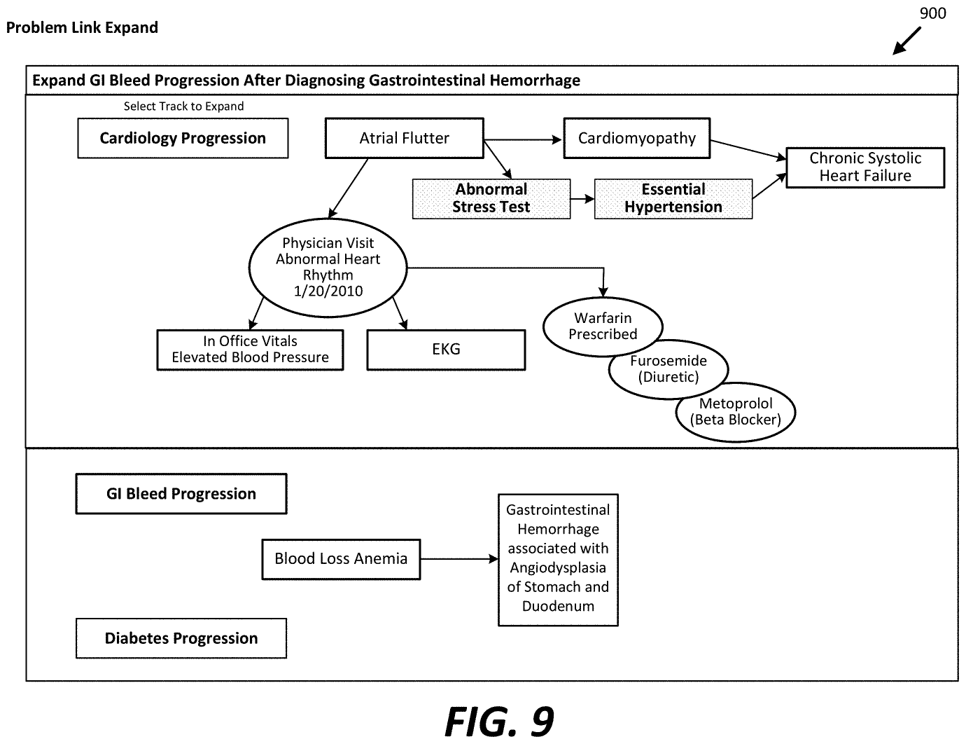

FIG. 9 shows interface 900, which is an expansion of interface 800. Interface 900 shows that the user can expand a different track or problem group. In interface 900, the application is not only providing the warning of an adverse effect (bleeding potentially due to Warfarin) but also triggering for a potential problem resolution or partial resolution. In this case, atrial flutter is potentially resolved by the pacemaker/ICD (implantable cardio defibrillator) implant. Whether this is full resolution of the problem or if it is sufficiently mitigated as demonstrated over time (e.g. 6 months), the trigger is then presented to drive other treatment modifications (e.g. discontinue warfarin). Displaying the trigger or note can be done on subsequent visits with or without the problem selection.

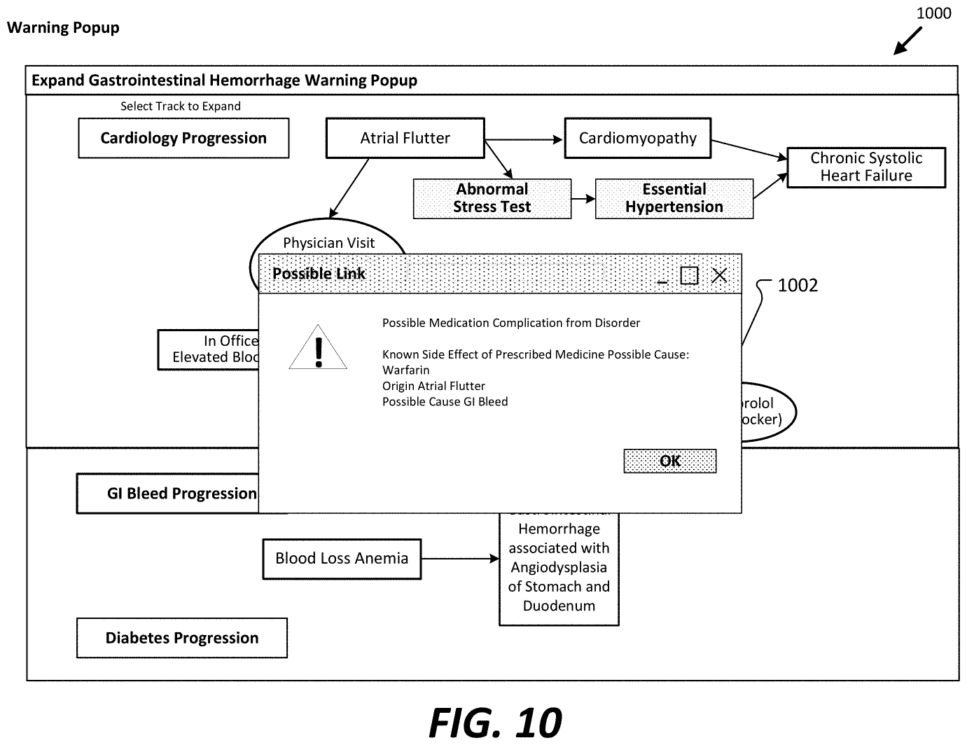

FIG. 10 shows interface 1000. Interface 1000 shows one possible way to notify a user regarding a determination made based on analysis of the electronic medical record data. In particular, interface 1000 provides a user with notice 1002 that there may be a connection, link, or side effect between multiple items in the dynamic health record problem list. Notice 1002 is a suggestion for the user to investigate and confirm on their own—it is not intended to provide clinical decision support. The cross-referencing becomes more important for patients with multiple chronic diseases where there may be more than a dozen issues in the problem list, more than a dozen prescribed drugs, and triggering events can be years apart.

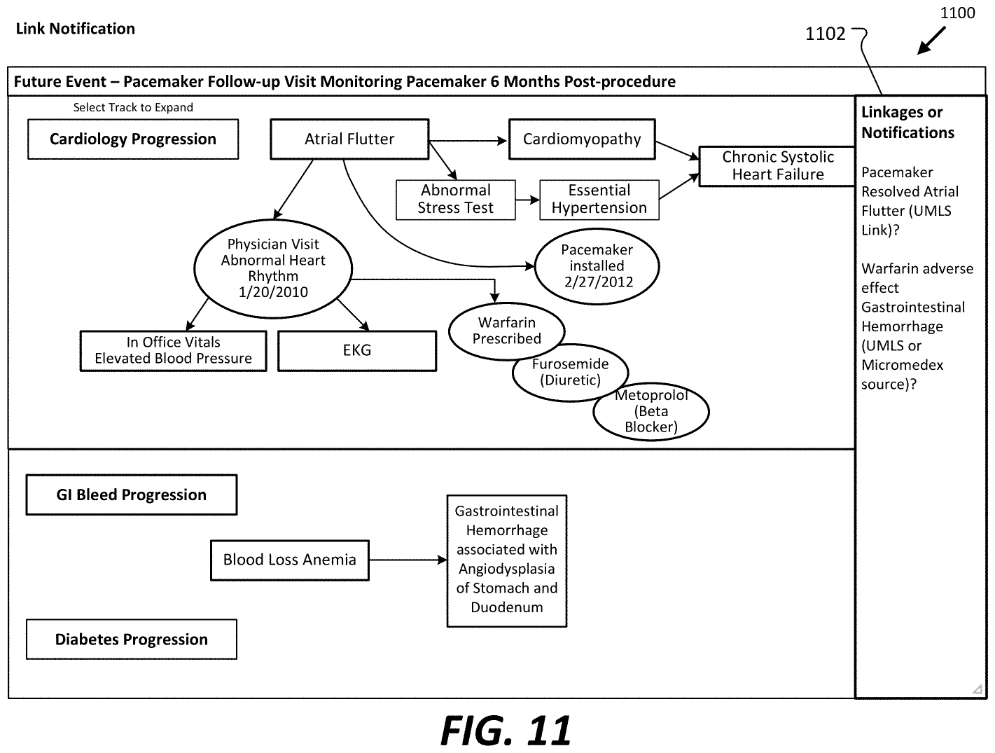

FIG. 11 shows interface 1100, which provides an alternative embodiment to interface 1000 for notifying a user regarding a determination. In particular, notice 1102 includes one or more determinations made based on analysis of the dynamic health record problem list. Notice 1102 references the source data that made the connection (e.g., UMLS, Micromedex, publication/literature search, etc.). In some instances, a user can select one or more references to drill into the reason for the linkage and explore the provenance of the issue. Similarly, other items in notice 1102 are selectable for a drill down to the source document and its provenance.

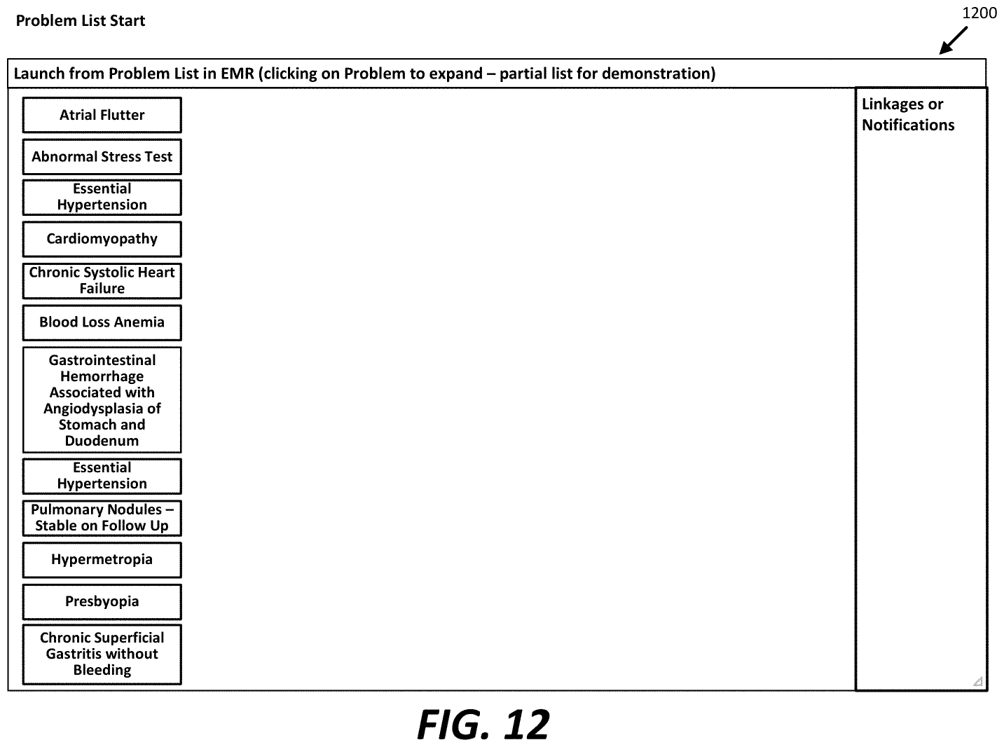

FIG. 12 shows interface 1200, which is an alternative starting point for a user. In some embodiments, interface 1200 is presented in an EMR/personal health record (PHR) problem list natively. Prior to display of interface 1200, a user selected a particular problem list item (atrial flutter) and interface 1200 is launched with this context (patient and problem. Alternatively, interface 1200 could have been launched from any of the linked items in the EMR/PHR such as a visit or visit note, medication (e.g., warfarin), and then interface 1200 launched with this context as the starting point.

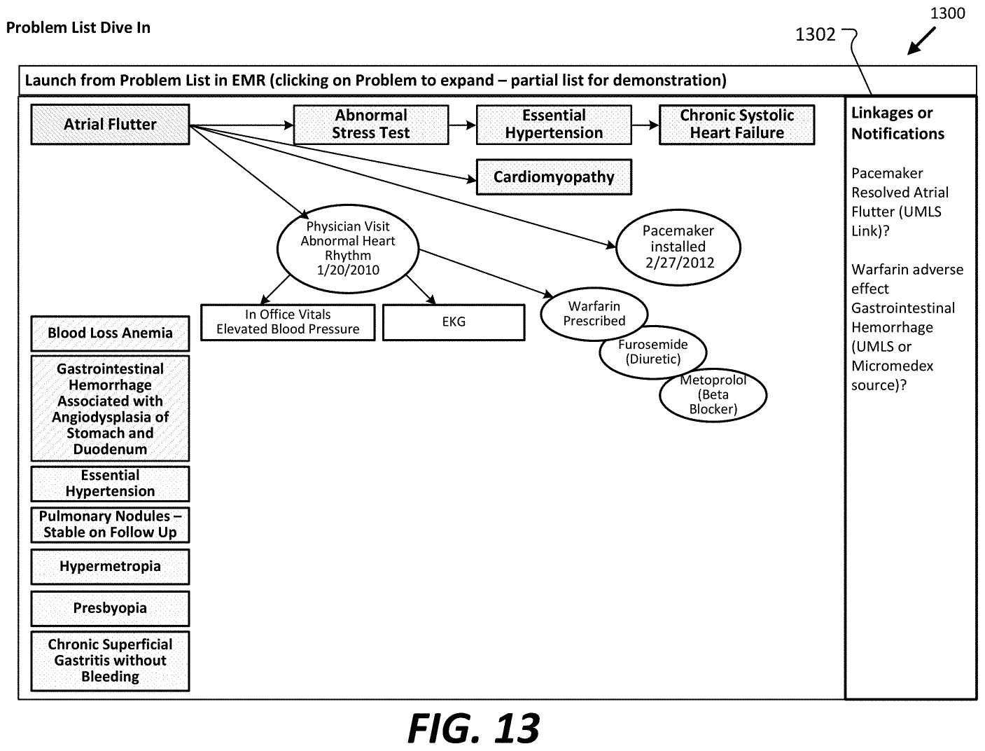

FIG. 13 shows interface 1300, which can be a subsequent display to interface 1200. In particular, interface 1300 can be displayed after a user expands on a particular item in the problem list to show both the progression and linkage within an ontology. Additionally, interface 1300 can show linked source data (e.g., physician visits, procedures, results, prescriptions, etc.). Interface 1300 also shows the linkages/notifications panel 1302 that includes possible connections, links, and/or side effects between multiple items in the dynamic health record problem lists shown on the screen. Additionally, interface 1300 can highlight items in the dynamic health record problem list identified in the linkages/notifications panel 1302, to facilitate reference by the user.

It is to be understood that the embodiments disclosed and contemplated herein are not limited to the details and the arrangement of components set forth in the description or illustrated in the accompanying drawings. Aspects of the disclosure are capable of other embodiments and of being practiced or of being carried out in various ways.

Also, it is to be understood that the phraseology and terminology used herein is for the purpose of description and should not be regarded as limiting. The use of “including,” “comprising” or “having” and variations thereof herein is meant to encompass the items listed thereafter and equivalents thereof as well as additional items. The terms “mounted,” “connected” and “coupled” are used broadly and encompass both direct and indirect mounting, connecting and coupling. Further, “connected” and “coupled” are not restricted to physical or mechanical connections or couplings, and may include electrical connections or couplings, whether direct or indirect. Also, electronic communications and notifications may be performed using any known means including direct connections, wireless connections, etc.

A plurality of hardware and software based devices, as well as a plurality of different structural components may be utilized to implement aspects of this disclosure. In addition, embodiments of the disclosure may include hardware, software, and electronic components or modules that, for purposes of discussion, may be illustrated and described as if the majority of the components were implemented solely in hardware. However, one of ordinary skill in the art, and based on a reading of this detailed description, would recognize that, in at least one embodiment, the electronic-based aspects of the disclosure may be implemented in software (for example, stored on non-transitory computer-readable medium) executable by one or more processors. As such, it should be noted that a plurality of hardware and software based devices, as well as a plurality of different structural components, may be utilized to implement the disclosure. For example, “mobile device,” “computing device,” and “server” as described herein may include one or more electronic processors, one or more memory modules including non-transitory computer-readable medium, one or more input/output interfaces, and various connections (for example, a system bus) connecting the components.

What is claimed is:

1. A system for generating a health record problem list, the system comprising:
an electronic processor; and
memory storing instructions that, when executed by the electronic processor, cause the system to:
obtain electronic health data;
apply natural language processing to generate extracted health data from the electronic health data;
map the extracted health data to normalized terms in one or more ontologies, thereby generating normalized health data;
determine if any relationship exists between the normalized health data, including:
identifying originating medical issue data; and
identifying subsequent relevant medical data;
when a relationship exists between the at least two medical record entries using an ontological list, generate a relationship map relating the originating medical data with the subsequent relevant medical data;
receive a request for dynamic health record problem list data from a client device; and
provide the dynamic health record problem list data to the client device, wherein the dynamic health record problem list data includes the relationship map.

2. The system according to claim 1, wherein the memory further stores instructions that, when executed by the electronic processor, cause the system to:
generate a relative ranking of items in the normalized health data, the relative ranking providing a treatment priority indication.

3. The system according to claim 1, wherein the memory further stores instructions that, when executed by the electronic processor, cause the system to:
generate a trust ranking of items in the normalized health data, the trust ranking providing a veracity indication.

4. The system according to claim 1, wherein the memory further stores instructions that, when executed by the electronic processor, cause the system to:
generate a recommendation based on the normalized health data.

5. The system according to claim 4, wherein the recommendation includes a suggested follow-up or a suggested clinical determination.

6. The system according to claim 5, wherein the recommendation includes a potential connection between items in the normalized health data.

7. The system according to claim 1, wherein obtaining electronic health data includes obtaining data from at least two unrelated medical record databases.

8. The system according to claim 1, wherein the memory further stores instructions that, when executed by the electronic processor, cause the system to:
de-duplicate a source of truth in the normalized health data.

9. The system according to claim 1, wherein the memory further stores instructions that, when executed by the electronic processor, cause the system to:
deprioritize a resolved issue in the normalized health data.

10. The system according to claim 1, wherein the memory further stores instructions that, when executed by the electronic processor, cause the system to:
link normalized health data to one or more source documents.

11. Non-transitory computer-readable medium including instructions that, when executed by an electronic processor, perform a set of functions, the set of functions comprising:
obtaining electronic health data;
applying natural language processing to generate extracted health data from the electronic health data;
mapping the extracted health data to normalized terms in one or more ontologies, thereby generating normalized health data;
determining if any relationship exists between the normalized health data, including:
identifying originating medical issue data; and
identifying subsequent relevant medical data;
when a relationship exists between the at least two medical record entries using an ontological list, generating a relationship map relating the originating medical data with the subsequent relevant medical data; and
generating a practice recommendation based on the normalized health data.

12. The non-transitory computer readable medium according to claim 11, wherein the set of functions further comprises:
receiving a request for patient adjusted health history data from a client device; and
providing the patient adjusted health history data to the client device, wherein the patient adjusted health history data includes the relationship map.

13. The non-transitory computer readable medium according to claim 12, wherein the set of functions further comprises:
generating a relative ranking of items in the normalized health data, the relative ranking providing a treatment priority indication; and
generating a trust ranking of items in the normalized health data, the trust ranking providing a veracity indication.

14. The non-transitory computer readable medium according to claim 13, wherein the practice recommendation includes a suggested follow-up or a suggested clinical determination; and
wherein obtaining electronic health data includes obtaining data from at least two unrelated medical record databases.

15. The non-transitory computer readable medium according to claim 13, wherein the set of functions further comprises:
de-duplicating a source of truth in the normalized health data; and
deprioritizing a resolved issue in the normalized health data.

16. The non-transitory computer readable medium according to claim 15, wherein the set of functions further comprises:
linking normalized health data to one or more source documents.

17. A method for generating an interactive patient data problem list with a server, the method comprising:
obtaining electronic health data;
applying natural language processing to generate extracted health data from the electronic health data;
mapping the extracted health data to normalized terms in one or more ontologies, thereby generating normalized health data;
determining if any relationship exists between the normalized health data, including:
identifying originating medical issue data; and
identifying subsequent relevant medical data;
when a relationship exists between the at least two medical record entries using an ontological list, generating a relationship map relating the originating medical data with the subsequent relevant medical data;
receiving a request for dynamic health record problem list data from a client device; and
providing the dynamic health record problem list data to the client device, wherein the dynamic health record problem list data includes the relationship map.

18. The method according to claim 17, wherein obtaining electronic health data includes obtaining data from at least two unrelated medical record databases.

19. The method according to claim 18, further comprising:
generating a practice recommendation based on the normalized health data, wherein the practice recommendation includes a suggested follow-up or a suggested clinical determination;
generating a relative ranking of items in the normalized health data, the relative ranking providing a treatment priority indication; and
generating a trust ranking of items in the normalized health data, the trust ranking providing a veracity indication.

20. The method according to claim 19, further comprising:
de-duplicating a source of truth in the normalized health data;
deprioritizing a resolved issue in the normalized health data; and
linking the normalized health data to one or more source documents.
